(19) United States
(12) Patent Application Publication
(10) Pub. No.: US 2003/0195161 A1
(43) Pub. Date: Oct. 16, 2003

(54) Composition comprising camptothecin or a camptothecin derivative and a topoisomerase II inhibitor for the treatment of cancer

(76) Inventor: Marie Christine Bissery, Vitry Sur Seine (FR)

Correspondence Address: FINNEGAN, HENDERSON, FARABOW, GARRETT & DUNNER, LLP, 1300 I STREET, NW, WASHINGTON, DC, 20005, US

(21) Appl. No.: 09/809,329

(22) Filed: Mar. 16, 2001

Related U.S. Application Data
(60) Provisional application No. 60/190,061, filed on Mar. 17, 2000.

(51) Int. Cl. A61K 31/7048; A61K 31/704; A61K 31/4741

(52) U.S. Cl. 514/27; 514/34; 514/280

(57) ABSTRACT

The present invention relates to therapeutic associations for the treatment of cancer, comprising an effective amount of a camptothecin, or a camptothecin derivative, with an effective amount of a topoisomerase II inhibitor, such as doxorubicin, daunorubicin, or etoposide, and methods of using such therapeutic associations.

The present application claims the benefit of U.S. Provisional Application No. 60/190,061 filed Mar. 17, 2000.

The present invention relates to therapeutic compositions comprising an effective amount of camptothecin, or a camptothecin derivative, in combination with an effective amount of a topoisomerase II inhibitor for the treatment of cancer.

European patent EP 137,145, specifically incorporated by reference herein, describes camptothecin derivatives of the formula:

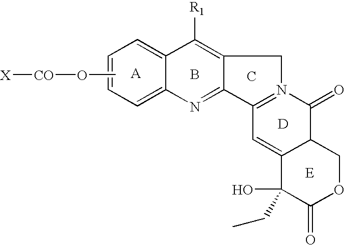

in which, in particular, R₁ is hydrogen, halogen or alkyl; X is a chlorine atom, or NR₂R₃, in which R₂ and R₃, which may be identical or different, may represent a hydrogen atom, an optionally substituted alkyl radical, a carbocycle or a heterocycle which are optionally substituted, or alkyl radicals (optionally substituted) forming, with the nitrogen atom to which they are attached, a heterocycle optionally containing another heteroatom chosen from O, S, and/or NR₄, wherein R₄ is a hydrogen atom or an alkyl radical; and in which the group X—CO—O— is located on ring A in position 9, 10, or 11.

These camptothecin derivatives are anticancer agents which inhibit topoisomerase I, among which irinotecan, in which X—CO—O— is [4-(1-piperidino-1-piperidino]carbonyloxy, is an active principle which is particularly effective in treatment of solid tumors. Camptothecin and camptothecin derivatives such as irinotecan (CPT-11) are cytotoxic alkaloids which possesses strong anti-tumor activities. Irinotecan shows clinical activity against colon, gastric, ovarian, and small cell lung cancers, as well as non-Hodgkin's lymphoma (Bissery, M. et al., Anti Cancer Drugs, 7:166-174 (1996)).

The European patent application EP 74,256 also describes other camptothecin derivatives which are also mentioned as anticancer agents, in particular, derivatives of a structure analogous to the structure given above and in which X—CO—O— is replaced with a radical —X′R′ for which X′ is O or S, and R′ is a hydrogen atom or an alkyl or acyl radical.

Other camptothecin derivatives have also been described, for example, in the following publications, patents, or patent applications: EP 56,692; EP 88,642; EP 296,612; EP 321,122; EP 325,247; EP 540,099; EP 737,686; WO 90/03169; WO 96/37496; WO 96/38146; WO 96/38449; WO 97/00876; U.S. Pat. No. 7,104,894; JP 57 116,015; JP 57 116,074; JP 59 005,188; JP 60 019,790; JP 01 249,777; JP 01 246,287; and JP 91 12070; Canc. Res., 38 (1997) Abstr. 1526 or 95 (San Diego, April 12-16); Canc. Res., 55(3):603-609 (1995); or AFMC Int. Med. Chem. Symp. (1997) Abstr. PB-55 (Seoul, Korea; July 27-August 1).

Camptothecin derivatives are usually administered by injection, more particularly intravenously in the form of a sterile solution or an emulsion. Camptothecin derivatives, however, can also be administered orally, in the form of solid or liquid compositions.

However, while camptothecin derivatives are considered as some of the most powerful substances possessing anti-tumor activity for colorectal cancers, the use of these compounds can be improved in clinical treatments by association with other antitumor agents.

Among such antitumor agents are topoisomerase II inhibitors, many of which possess antineoplastic properties. Some of these agents belong to the class of anthracycline antibiotics, such as daunorubicin, doxorubicin, annamycin, epirubicin, mitomycin, bleomycin, idarubicin (idamycin), and cororubicin. Other agents belong to the class of epipodophyllotoxins, such as etoposide (VP-16), and teniposide (VM-26).

The combination of CPT-11 and the anthracycline antibiotic, doxorubicin, has been studied in Japan (Furuta, Tomio et al., Cancer Chemotherapy, 18(3): 393-402 (1991)). In that study, however, the evaluation of the combination was only conducted on L1210 mouse leukemia, not solid tumors. The route of administration of irinotecan and doxorubicin was via the abdominal cavity, that is, the drugs were administered intraperitoneally and not orally or intravenously. Furthermore, that study did not evaluate the effect of the highest non-toxic dose of either camptothecin or doxorubicin as single agents. Without such a determination, it is not possible to determine the synergistic effect of the CPT-11/doxorubicin combination.

It has now been found that the combination of CPT-11 with a topoisomerase II inhibitor such as doxorubicin or etoposide is more active at a lower dose than the highest non-toxic dose of each single agent for the treatment of cancer, for example, in the treatment of pancreatic ductal adenocarcinoma and mammary adenocarcinoma.

The efficacy of a combination may be demonstrated by determination of therapeutic synergy. A combination manifests therapeutic synergy if it is therapeutically superior to one or other of the constituents used at its optimum dose (T. H. Corbett et al., Cancer Treatment Reports, 66: 1187 (1982)).

The efficacy of a combination may also been demonstrated by comparison of the maximum tolerated dose of the combination with the maximum tolerated dose of each of the separate constituents in the study in question. This efficacy may be quantified, for example by the log₁₀ cell kill, which is determined by the following formula:

log₁₀ cell kill=T−C(days)/3.32×T_{d}

in which T−C represents the time taken for the cells to grow, which is the mean time in days for the tumors of the treated group (T) to reach a predetermined value (1 g for example) and the tumors of the control group (C) to reach the same value, and T_{d} represents the time in days needed for the volume of the tumors in the control group to double. (T. H. Corbett et al., Cancer, 40: 2660-2680 (1977); F. M. Schabel et al., Cancer Drug Development, Part B, Methods in Cancer Research, 17: 3-5 1, New York, Academic Press Inc. (1979)). A product is considered to be active if the log₁₀ cell kill is greater than or equal to 0.7. A product is considered to be very active if the log₁₀ cell kill is greater than 2.8.

It has now been found that administration of CPT-11 in combination with doxorubicin in the following manner with the following schedules results in a combination that is synergistically active against pancreatic ductal adenocarcinoma, that is, the maximum tolerated dose of the CPT-11/doxorubicin combination is therapeutically superior to the maximum tolerated dose of either CPT-11 or doxorubicin alone.

The products may be administered simultaneously, semi-simultaneously, separately, or spaced out over a period of time so as to obtain the maximum efficacy of the combination. As a result, the invention is not limited to the compositions obtained by the physical association of the drugs, but also include those which permit separate administration, either simultaneously, semi-simultaneously, or spaced out over a period of time.

### BRIEF DESCRIPTION OF THE DRAWING

FIG. 1 presents a table evaluating irinotecan (CPT-11), doxorubicin, and the combination thereof as therapeutics against pancreatic ductal adenocarcinoma in a murine model system.

### EXAMPLE 1

Irinotecan alone was tested in various murine models is indicated in Table I. The efficacy was measured by the log₁₀ cell kill (LCK). The optimal total dose for oral and intravenous administration routes was also determined.

TABLE 1
Comparison of Oral and I.V. Irinotecan Administration

| Tumor | | | Optimal Total | |
|---|---|---|---|---|
| (mice) | Route | Schedule days | Dose mg/kg | LCK |
| C51 | oral | 5, 7, 9, 13, 15, twice daily* | 845 | 2.5 |
| (BALB/c) | i.v. | 5, 7, 9, 13, 15, twice daily* | 615 | 3.0 |
| C26 | oral | 3-7 twice daily* | 558 | 0.9 |
| (BALB/c) | i.v. | twice daily* | 228 | 0.7 |
| P03 | oral | twice daily* | 900 | 3.4† |
| (B6D2F1) | i.v. | twice daily* | 346.2 | 3.2† |
| MA16/C | oral | 5-9 | 230.5 | 2.7 |
| (C3H/ | i.v. | 5-9 | 130.5 | 2.6 |
| HeN) | | | | |
| GOS | oral | 3-7, twice daily* | 900 | 2.1 |
| (B6D2F1) | i.v. | 3, 5, 7 twice daily* | 346.2 | 2.2 |

*The two administrations were 4 hours apart.
†1/5 tumor free survivor on day 120.

Both methods of administration, oral and intravenous, resulted in similar tolerance, as measured by body weight loss (8.5%), nadir (7 days post last administration), and recovery (5 days post nadir, i.e., 12 days post first administration). This study showed that the efficacy in tumor bearing mice was similar for oral and i.v. irinotecan administration across all five tumor models tested in three different mice strains. The maximum tolerated oral dose for irinotecan was shown to be about 1.4 to 2.6 times the maximum tolerated i.v. dose.

### EXAMPLE 2

Cross-resistance between various agents was measured in murine leukemia cell lines. P388/CPT is a camptothecin-resistant leukemia that was established in vitro (Biochem. Pharmacol., 45: 339 (1993)) and maintained in vivo by i.p. passages in DBA2 female mice. The chemosensitivity of i.p. P388/CPT was evaluated with i.v. P388 sensitive reference drugs with different mechanisms of action. Antitumor efficacy was determined at the highest non-toxic dose as percent increase in life span (ILS), where:

ILS=100×[(median day of death (MDD) of treated mice)−(MDD control mice)]−(MDD control mice)

A minimal level of activity equals an ILS of greater than 26%. P388/CPT was found resistant to camptothecin s.c. and CPT-11, but sensitive to both doxorubicin and etoposide. These results show that this cell line was still sensitive to topoisomerase II inhibitors even when camptothecin resistance was present (Vrignaud, P. et al., Proc. Amer. Assoc. Cancer Res., 35: 363, Abstract No. 2163 (1994)). Table II tabulates the results from this study.

TABLE II

| Agents/% ILS | P388 | P388/CPT (TFS) | Comment |
|---|---|---|---|
| CPT (sc) | 82 | 0 | resistant |
| CPT-11 (i.v.) | 91 | 0 | resistant |
| doxorubicin (i.v.) | 122 | 180 | sensitive |
| etoposide (i.v.) | 127 | 121 | sensitive |

### EXAMPLE 3

Simultaneious intravenous administration of irinotecan (CPT-11) and representative topoisomerase II inhibitors was evaluated and the results are shown in Table III.

TABLE III

| CPT-11 | | | % HNTD of | Host recovery | Therapeutic |
|---|---|---|---|---|---|
| plus: | Tumor site | Schedule | single agents | (days) | response |
| doxorubicin | P03 sc | simult. | 65 | 13 | ≧ |
| etoposide | MA16/C sc | simult. | 60 | 6 | = |

HNTD represents the highest nontoxic dose.
≧: Better dose response for the combination.
=: Dose response for combination about equal to each agent alone.

Simultaneous administration of CPT-11 and a topoisomerase II inhibitor resulted in a therapeutic response that for the etoposide combination was about equal to the agents alone. For the doxorubicin combination, the therapeutic response was better than for the agents alone.

### EXAMPLE 4

The effect of the combination of CPT-11 and doxorubicin was evaluated a three-arm study in mice bearing pancreatic ductal adenocarcinoma PO3. In the first arm, three doses of CPT were given orally on days six through nine, twice a day. In the second arm, three doses of doxorubicin were given intravenously on days six and nine. In the combination third arm, five dosage levels of CPT-11 were administered orally on days six through nine, twice a day, with administration of five dosage levels of doxorubicin intravenously on days six and nine. This third arm illustrated an example of semi-simultaneous administration. In all three arms, the two daily administrations of CPT-11 were given four hours apart.

The results obtained in the study of single agents CPT-11 and doxorubicin and the combination CPT-11/doxorubicin are given in Table IV.

TABLE IV
Evaluation of CPT-11 in Combination with Doxorubicin Against Pancreatic Ductal Adenocarcinoma P03
on B6D2F₁ Female Mice CM-887

| | | | | Log₁₀ | | Time for median | |
|---|---|---|---|---|---|---|---|
| | | Dosage | Schedule | cell | T-C | tumor to reach | |
| Agent | Route | (mg/kg/adm) | (days) | kill | (days) | 1000 mg in days | Comments |
| CPT-11 | p.o.; 0.2 ml | 100.8 | 6-9 (twice/day) | 1.3 | 9.8 | 26.2 | HDT active |
| CPT-11 | p.o.; 0.2 ml | 62.5 | 6-9 (twice/day) | 1.2 | 9.3 | 25.7 | Active |
| CPT-11 | p.o.; 0.2 ml | 38.8 | 6-9 (twice/day) | 0.5 | 4.0 | 20.4 | Inactive |
| Doxorubicin | i.v.; 0.2 ml | 10.0 | 6, 9 | —* | —* | —* | HDT highly active |
| Doxorubicin | i.v.; 0.2 ml | 6.2 | 6, 9 | 1.2 | 9.5 | 25.9 | Active |
| Doxorubicin | i.v.; 0.2 ml | 3.8 | 6, 9 | 1.0 | 7.5 | 23.9 | Active |
| Doxorubicin + | i.v., 0.2 ml | 5.0 | 6, 9 | —* | —* | —* | HDT highly active |
| CPT-11 | p.o., 0.2 ml | 80.6 | 6-9 (twice/day) | | | | |
| Doxorubicin + | i.v., 0.2 ml | 4.1 | 6, 9 | 4.7 | 35.6 | 52.0 | Highly active |
| CPT-11 | p.o., 0.2 ml | 65.5 | 6-9 (twice/day) | | | | |
| Doxorubicin + | i.v., 0.2 ml | 3.1 | 6, 9 | 3.1 | 23.5 | 39.9 | Highly active |
| CPT-11 | p.o., 0.2 ml | 50.4 | 6-9 (twice/day) | | | | |
| Doxorubicin + | i.v., 0.2 ml | 2.2 | 6, 9 | 1.6 | 12.3 | 28.7 | Active |
| CPT-11 | p.o., 0.2 ml | 35.3 | 6-9 (twice/day) | | | | |
| Doxorubicin + | i.v., 0.2 ml | 1.2 | 6, 9 | 0.5 | 4.1 | 20.5 | Inactive |
| CPT-11 | p.o., 0.2 ml | 20.1 | 6-9 (twice/day) | | | | |

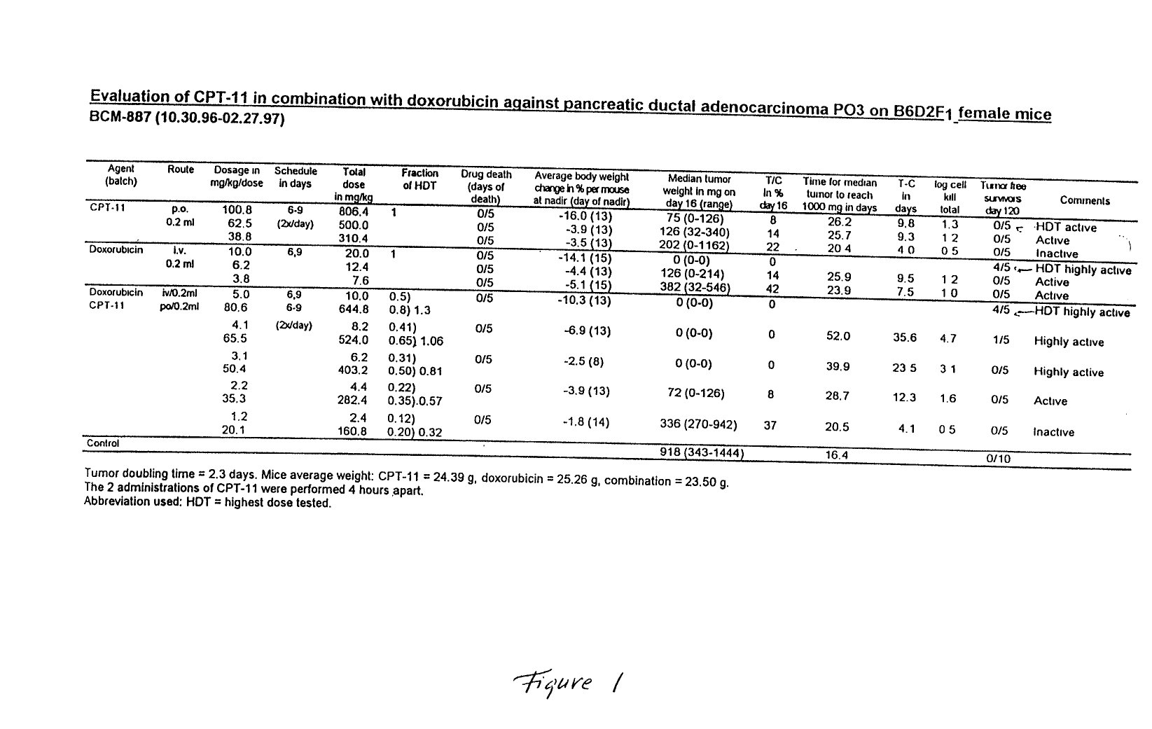

HDT: highest dose tested;
T-C: Tumor growth delay;
p.o.: per os;
i.v.: intravenous
*There are no values presented because 4 out of 5 animals tested were tumor free survivors.
The data comprising this table were compiled from FIG. 1.

While irinotecan as a single agent was active at two dosages, it was not highly active. Doxorubicin alone and the doxorubicin/CPT-11 combination were both highly active. However, the combination was highly active at a much lower dosage than the single agents. For example, in the highly active combination dosage doxorubicin was present at 50% of the highly active single agent dose, and CPT-11 was present at 80% of the merely active dose. Further, the combination was highly active over a larger dosage range than either of the single agents, i.e., four of the five combination dosages tested were at least active, and three were even highly active. The combination allowed for much lower dosages, while maintaining activity. Therefore, the combination of CPT-11 and doxorubicin was synergistically active for the treatment of cancer.

### EXAMPLE 5

The effectiveness of irinotecan combination chemotherapy methods was tested in a dose response study in a murine tumor model. Three arms were evaluated for tolerance and efficacy. Tolerance was measured by mortality, body weight loss at nadir, host recovery time, and combination toxicity index. Efficacy end points for solid tumor models were tumor growth delay (T/C), log₁₀ cell kill (LCK, defined above), tumor regressions (i.e., complete remission (CR),or partial remission (PR)).

Combination toxicity index (CTI) was calculated as the sum of the fraction of LD₁₀'s for each agent used in each combination (Cancer Treatment Reports, 66(5); 1187-1200 (1982)). The LD₁₀ for the single agent was obtained by plotting the toxicity of that agent and the dosage in mg/kg as a log probit graph. Subsequently, the CTI LD₁₀ was obtained by plotting as a log probit graph the observed lethality and the corresponding CTI calculated as the sum of the fraction of the LD₁₀ of each single agent. When the CTI equals one, only 50% of the LD₁₀'s of each agent can be used in combination without additional toxicity, and when the CTI equals two, 100% of the LD₁₀'s of each agent can be used in combination without additional toxicity.

Table V compares different application methods for representative topoisomerase II inhibitors alone and in combination, i.e., i.v. or per os (p.o.), as indicated.

TABLE V

| | | Schedule | HNTD | | | |
|---|---|---|---|---|---|---|
| Agents | Tumor site | days | Dose mg/kg | | LCK | CTI |
| CPT-11, p.o. | P03, sc | 6-9, | 806 | — | 1.3 | |
| | | twice | | | | |
| | | daily | | | | |
| doxorubicin, i.v. | | 6, 9 | — | 20.0 | —* | |
| combination | | | 644.8 | 10.0 | —* | ≅1.3 |
| CPT-11, p.o. | MA16/C, sc | 3-7 | 198.5 | — | 2.0 | |
| etoposide, i.v. | | 3, 5, 7 | — | 90.0 | 2.2 | |
| combination | | | 144.0 | 40.5 | 2.1 | ≅1.2 |

HNTD represents the highest nontoxic dose.
*4/5 tumor free survivors; the combination gave a superior dose response (efficacy at 4 dose levels)

This study confirmed the positive results obtained in Example 1. Irinotecan combined with doxorubicin at its highest non toxic dose resulted in 4/5 tumor free survivors. Irinotecan combined with etoposide gave no antagonist activity, and was more effective than CPT-11 alone. The CPT-11/etoposide combination at its highest non-toxic dose produced a log₁₀ cell kill of 2.1, which was superior to the log₁₀ cell kill of the highest non-toxic dose of CPT-11 as a single agent. The combination was therefore therapeutically superior to both of the single agents used at its optimum dose.

In conclusion, a combination of a topoisomerase II inhibitor, such as an anthracycline antibiotic, i.e., doxorubicin, daunorubicin, annamycin, or cororubicin, or an epipodophyllotoxin, i.e., etoposide (VP-16), or teniposide (VM-26), with irinotecan or other camptothecin derivative, is a highly active pharmaceutical composition and represents a new method for treating cancer.

I claim:

1. A therapeutic pharmaceutical composition, comprising an effective amount of camptothecin, or a camptothecin derivative, in combination with an effective amount of a topoisomerase II inhibitor for the treatment of solid tumors.

2. A therapeutic pharmaceutical composition, comprising an effective amount of CPT-11, in combination with an effective amount of a topoisomerase II inhibitor for the treatment of solid tumors.

3. The composition according to one of claims 1 or 2, wherein said topoisomerase II inhibitor is an anthracycline antibiotic.

4. The composition according to claim 3, wherein said antibiotic is doxorubicin.

5. The composition according to claim 3, wherein said antibiotic is daunorubicin.

6. The composition according to one of claims 1 or 2, wherein said topoisomerase II inhibitor is an epipodophyllotoxin.

7. The composition according to claim 6, wherein said epipodophyllotoxin is etoposide.

8. The composition according to claim 6, wherein said epipodophyllotoxin is teniposide.

9. The composition according to one of claims 1 or 2, wherein said solid tumor is a mammary adenocarcinoma.

10. The composition according to one of claims 1 or 2, wherein said solid tumor is a pancreatic ductal adenocarcinoma.

11. A synergistic therapeutic pharmaceutical composition, comprising an effective amount at least two agents, wherein at least one agent is CPT-11, in combination with an effective amount of at least one second agent, wherein said second agent is doxorubicin, for the treatment of solid tumors.

12. The composition according to claim 11, wherein the at least two agents are administered simultaneously, semi-simultaneously, or separately.

13. A method of treating a solid tumor, comprising administering an effective amount of camptothecin, or a camptothecin derivative, as a first agent, in combination with administration of an effective amount of a topoisomerase II inhibitor as a second agent, wherein the agents are administered simultaneously, semi-simultaneously, or separately.

14. The method according to claim 13, wherein the camptothecin derivative is CPT-11, and the topoisomerase II inhibitor is an anthracycline antibiotic.

15. The method according to claim 14, wherein said antibiotic is doxorubicin.

16. The method according to claim 14, wherein said antibiotic is daunorubicin.

17. The method according to claim 13, wherein the camptothecin derivative is CPT-11, and the topoisomerase II inhibitor is an epipodophyllotoxin.

18. The method according to claim 17, wherein said epipodophyllotoxin is etoposide.

19. The method according to claim 17, wherein said epipodophyllotoxin is teniposide.

20. The method according to any one of claims 13-19, wherein the camptothecin derivative is administered orally.
